(19) United States
(12) Patent Application Publication
(10) Pub. No.: US 2023/0366893 A1
(43) Pub. Date: Nov. 16, 2023

(54) METHODS TO DETERMINE RISK OF NEUROTOXICITY

(71) Applicant: WASHINGTON UNIVERSITY, St. Louis, MO (US)

(72) Inventors: Omar Butt, St. Louis, MO (US); Armin Ghobadi, St. Louis, MO (US); Beau Ances, St. Louis, MO (US); John DiPersio, St. Louis, MO (US)

(73) Assignee: Washington University, St. Louis, MO (US)

(21) Appl. No.: 18/029,210

(22) PCT Filed: Sep. 29, 2021
(86) PCT No.: PCT/US21/52677

§ 371 Date: Mar. 29, 2023

Related U.S. Application Data
(60) Provisional application No. 63/084,667, filed on Sep. 29, 2020.

(51) Int. Cl. G01N 33/68

(52) U.S. Cl. CPC G01N 33/6896; G01N 2800/52; G01N 2800/50; G01N 2800/7047

(57) ABSTRACT

Among the various aspects of the present disclosure is the provision of methods to predict the risk of developing immunotherapy-associated neurotoxicity in a subject by measuring neurofilament light chain (NfL) levels in a biological sample obtained from the subject, wherein the subject is receiving or may receive an immunotherapy such as CAR T cell therapy.

### CROSS-REFERENCE TO RELATED APPLICATIONS

This application claims priority from U.S. Provisional Application Ser. No. 63/084,667 filed on 29 Sep. 2020, which is incorporated herein by reference in its entirety.

### STATEMENT REGARDING FEDERALLY SPONSORED RESEARCH OR DEVELOPMENT

Not applicable.

### MATERIAL INCORPORATED-BY-REFERENCE

Not applicable.

### FIELD OF THE INVENTION

The present disclosure generally relates to determining the risk of neurotoxicity.

### SUMMARY OF THE INVENTION

Among the various aspects of the present disclosure is the provision of methods to predict the risk of developing immunotherapy-associated neurotoxicity in a subject and methods of treatment thereof.

An aspect of the present disclosure provides for a method of detecting a neurofilament light chain (NfL) level in a biological sample comprising: obtaining or having previously obtained a biological sample from a subject; or detecting or measuring the neurofilament light chain (NfL) level in the biological sample. In some embodiments, biological sample is obtained or previously obtained from a subject at risk for developing an immunotherapy-associated neurotoxicity or the subject is in need of immunotherapy. In some embodiments, the detecting or measuring a neurofilament light chain (NfL) level in the biological sample is performed up to 30 days before the subject is expected to receive immunotherapy. Another aspect of the present disclosure provides for a method to determine if a subject is at risk for developing an immunotherapy-associated neurotoxicity comprising: obtaining or having previously obtained a biological sample from the subject; or detecting or measuring a neurofilament light chain (NfL) level in the biological sample. In some embodiments, if the subject has an elevated NfL level compared to a control or more than about 44 pg/mL, the subject is determined to be at higher risk for developing neurotoxicity. In some embodiments, if the subject has a normal level of NfL compared to a control or less than about 44 pg/mL, the subject is determined to be at normal or lower risk for developing neurotoxicity. In some embodiments, the detecting or measuring a neurofilament light chain (NfL) level in the biological sample is performed up to 30 days before the subject is expected to receive immunotherapy. Another aspect of the present disclosure provides for a method of treating or preventing an immunotherapy-associated neurotoxicity in a subject comprising: obtaining or having previously obtained a biological sample from the subject; detecting or measuring a neurofilament light chain (NfL) level in the biological sample; or administering a therapy for the immunotherapy-associated neurotoxicity if the subject has an elevated NfL level compared to a control or more than about 44 pg/mL before, after, or during immunotherapy. In some embodiments, the detecting or measuring a neurofilament light chain (NfL) level in the biological sample is performed up to 30 days before the subject is expected to receive immunotherapy. In some embodiments, the method of detecting NfL has a sensitivity of about or at least about 0.91 or specificity of about or at least about 0.95. In some embodiments, the immunotherapy-associated neurotoxicity is immune effector cell-associated neurotoxicity syndrome (ICANS). In some embodiments, the biological sample is or comprises serum, plasma, or cerebrospinal fluid (CSF). In some embodiments, the method further comprises contacting the biological sample with an anti-NfL antibody. In some embodiments, the NfL level is detected using an immunoassay optionally selected from Single Molecule Array (SiMoA), enzyme linked immunosorbent assay (ELISA), electrochemiluminescence (ECL), or electrochemiluminescence immunoassay (ECLIA). In some embodiments, the subject will, has, or is receiving an immunotherapy. In some embodiments, the subject is administered an immunotherapy. In some embodiments, the subject is administered an immunotherapy or the immunotherapy is a CAR T cell therapy optionally selected from engineered CAR T, universal allogeneic CAR T, CD19-specific CAR T, anti-CD19 CAR T cells, or anti-BCMA CAR T cells; anti-CD19 CAR T cells or anti-BCMA CAR T cells selected from axicabtagene ciloleucel, tisagenlecleucel, brexucabtagene autoleucel, lisocabtagene maraleucel, idecabtagene vicleucel, or other FDA approved CAR T therapy; the CAR T cell therapy is selected from non-FDA-approved or experimental CAR T cell therapy selected from anti-CD22, anti-CAIX, anti-PSMA, anti-MUC1, anti-FRα, anti-meso-RNA, anti-CEA, anti-IL13Rα2, anti-HER2, or universal allogenic CAR T cells; or combinations thereof. In some embodiments, the subject is administered an immunotherapy or the immunotherapy is a bispecific antibody therapy or a bispecific monoclonal antibody (BsMAb) therapy optionally selected from blinatumomab, emicizumab, or amivantamab. In some embodiments, the subject is administered an immunotherapy or the immunotherapy is an immune effector cells (IEC) therapy optionally selected from dendritic cells, natural killer (NK) cells, T cells, B cells, NK-CAR, CAR T, mesenchymal stem cells, genetically engineered CAR-T cells, therapeutic vaccines, or combinations thereof. In some embodiments, the subject is administered an immunotherapy or the immunotherapy is a non-IEC therapy or T cell engaging therapy optionally selected from a bispecific antibody, CD19-specific T cell engager, such as blinatumomab, T cell engaging monoclonal antibody, or bispecific T cell engager (BiTE) therapy. In some embodiments, the NfL level is detected in the biological sample obtained from the subject before, during, or after treatment with an immunotherapy. In some embodiments, the NfL level is detected in the biological sample obtained from the subject on multiple days or at multiple time points before or after the subject has been administered an immunotherapy. In some embodiments, the NfL level is measured before the subject is administered an immunotherapy. In some embodiments, the NfL level is measured at least once after the subject is administered an immunotherapy. In some embodiments, the NfL level is measured before or after the subject is administered an immunotherapy. In some embodiments, the NfL level is monitored in the subject before, during, or after an immunotherapy, or combinations thereof. In some embodiments, the NfL level is monitored for at least about 30 days before or after the subject is administered immunotherapy. In some embodiments, if a subject has elevated NfL or is determined to be at risk for ICANS, the subject is administered a therapeutic agent for immunotherapy-associated immunotoxicity within about 72 hours, or between about 3 days or about 10 days after receiving immunotherapy. In some embodiments, the subject has elevated levels of NfL or is determined to be at risk for ICANS, a steroidal anti-inflammatories selected from steroids, glucocorticoids, or corticosteroids optionally selected from beclomethasone; betamethasone; budesonide; cortisone; dexamethasone; hydrocortisone; methylprednisolone; prednisolone; prednisone; triamcinolone; or combinations thereof are administered before, during, or after immunotherapy. In some embodiments, if the subject has elevated levels of NfL or is determined to be at risk for ICANS, an antiseizure drug therapy or antiepileptic medication optionally selected from levetiracetam, lacosamide, benzodiazepines, valproic acid, carbamazepine, ethosuximide, gabapentin, lamotrigine, oxcarbazepine, phenytoin, topiramate, zonisamide, valproate, phenobarbital, lacosamide, or combinations thereof are administered before, during, or after immunotherapy. In some embodiments, the subject has, is at risk for, or is suspected of having cancer. In some embodiments, the subject has refractory cancer. In some embodiments, the subject has one or more of risk factors selected from the group consisting of: younger patient age; preexisting neurologic or medical comorbidities; high disease burden of an underlying malignancy; increased intensity of lymphodepleting therapy or cytopenias; or early or severe cytokine release syndrome (CRS) with high levels of inflammatory cytokines. In some embodiments, the subject has active central nervous system (CNS) disease. In some embodiments, the subject has, at risk for, or is suspected of having an immune effector-associated neurotoxicity or has symptoms selected from immune effector cell-associated encephalopathy (ICE), mild alterations in a level of consciousness to varying degrees of neurologic dysfunction, encephalopathy with confusion, behavioral changes, visual or auditory hallucinations, language dysfunction, speech alterations, apraxia, headache, fatigue, tremors, dysgraphia or other fine motor impairment, clinical or subclinical seizures, including status epilepticus, cerebral edema with coma, inattention or language deficits, aphasia, dysphasia, impaired fine motor skills, difficulty with word finding, confusion, somnolence; motor weakness, seizures, cerebral edema, coma, dysgraphia, lethargy, obtundation, stupor, limb weakness or numbness, loss of memory or vision, headache, cognitive or behavioral problems, sexual dysfunction, mild confusion, seizures, multifocal strokes, diffuse brain swelling (cerebral edema), or intracerebral hemorrhage. In some embodiments, the method further comprises monitoring the subject for development of cytokine release syndrome (CRS) after administering immunotherapy. In some embodiments, the method further comprises detecting elevated levels of inflammatory cytokines optionally selected from interferon gamma (IFN-gamma), IL-2, IL-6, IL-8, IL-10, IL-15, granulocyte colony-stimulating factor (G-CSF), granulocyte-macrophage colony-stimulating factor (GM-CSF), monocyte chemoattractant protein 1 (MCP-1), interferon gamma-induced protein 10 (IP-10), markers of astrocyte injury optionally selected from glial fibrillary acidic protein (GFAP), S100 calcium-binding protein B (S100B)), tumor necrosis factor alpha (TNF-alpha); or neurotoxic substances optionally selected from glutamate or quinolinic acid. In some embodiments, the method further comprises detecting elevated levels of lactate dehydrogenase (LDH) levels (as a marker of disease burden), significant thrombocytopenia (as a marker of bone marrow toxicity in heavily pretreated patients), rising inflammatory markers optionally selected from ferritin, C-reactive protein (CRP), or erythrocyte sedimentation rate (ESR; as evidence of cytokine release or immune activation).

Other objects and features will be in part apparent and in part pointed out hereinafter.

### DESCRIPTION OF THE DRAWINGS

Those of skill in the art will understand that the drawings, described below, are for illustrative purposes only. The drawings are not intended to limit the scope of the present teachings in any way.

FIG. 1A is a graph showing elevated blood neurofilament light chain (NfL) levels prior to CAR T transfusion (pre-infusion) predicts the development of immune effector cell-associated neurotoxicity syndrome (ICANS). Plasma banked prior to CAR T transfusion was compared between patients who ultimately developed severe (Grade III or higher) ICANS to those who never developed symptoms with an excellent ROC curve classification (AUC of 0.96) with a cut-off of 44 pg/mL. FIG. 1A shows baseline (pre-infusion) levels of NfL in patients who develop Grade 0 ICANS, Grade 1-2 ICANS, and Grade 3+ ICANS.

FIG. 1B is a graph comparing baseline (pre-infusion) NfL levels in plasma banked prior to CAR T transfusion between patients who ultimately developed ICANS to those who did not. FIG. 1B is the associated ROC curve showing excellent diagnostic capability with an AUC of 0.96, sensitivity of 0.91, and specificity of 0.95 of patients who developed any grade ICANS (1+) vs grade 0 for baseline NfL and post-infusion day 1 (D1) markers.

FIG. 10 is a heat map showing that elevated baseline (pre-infusion) NfL levels are highly correlated with ICANS grade (correlation between pre-treatment factors and pre-infusion biomarkers). All significant relationships after correction for multiple comparisons using false discovery rate (FDR) are outlined (*).

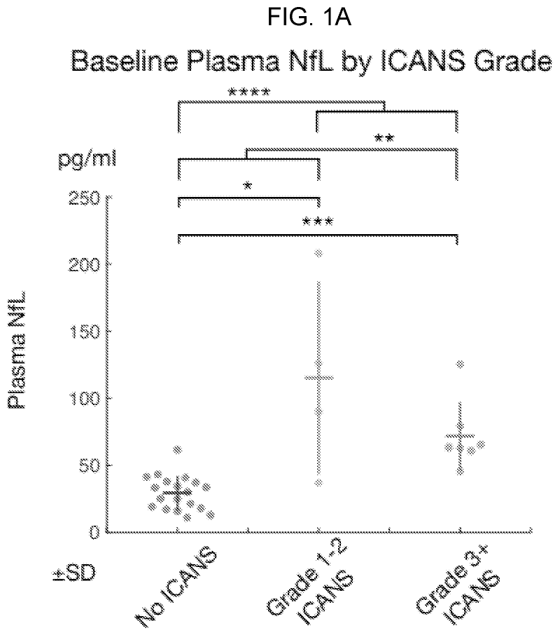

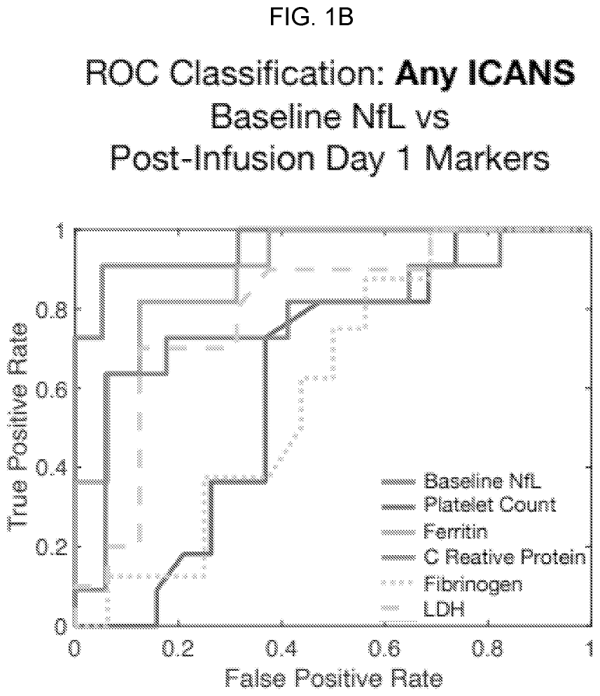

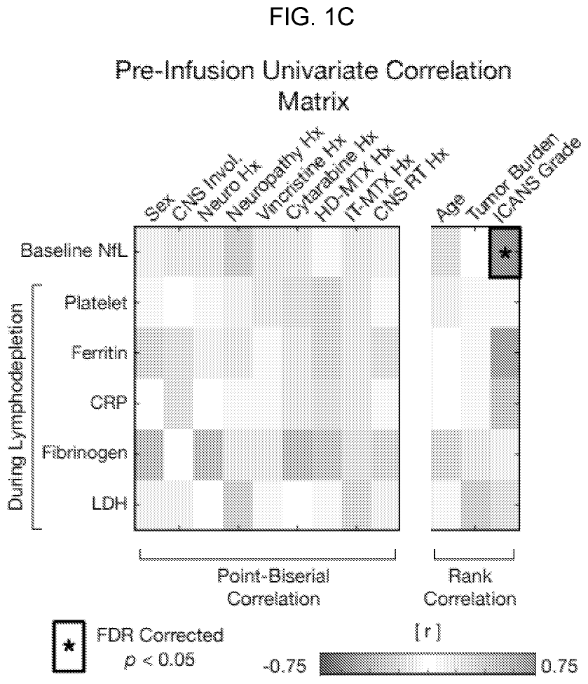

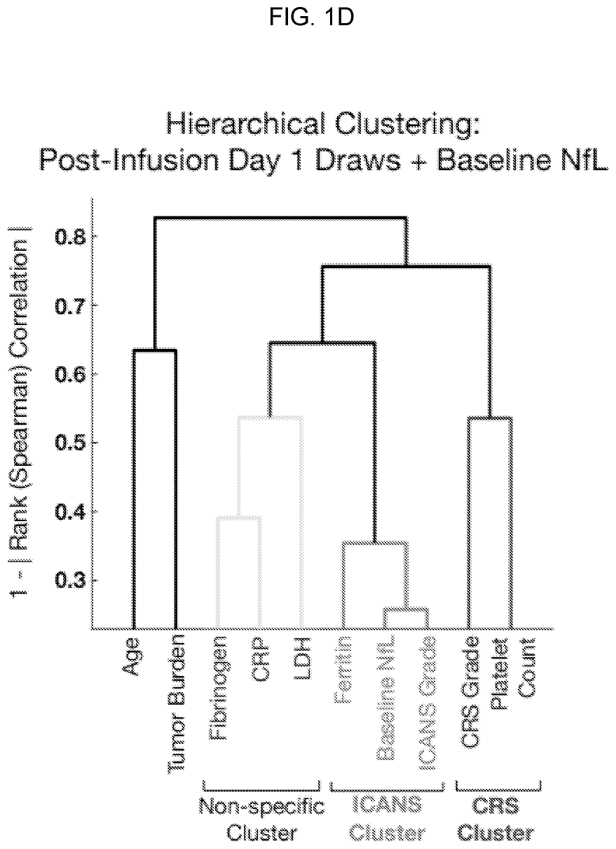

FIG. 1D is hierarchical clustering also showing the tight clustering of NfL levels with ICANS grade (correlation between pre-treatment factors and pre-infusion biomarkers). All significant relationships after correction for multiple comparisons using false discovery rate (FDR) are outlined (*).

### DETAILED DESCRIPTION OF THE INVENTION

The present disclosure is based, at least in part, on the discovery that certain neurofilament light chain (NfL) levels in humans indicate that a subject is at risk for developing neurotoxicity in response to immunotherapy. As shown herein, a simple blood-based test measuring NfL levels in plasma can be used to predict which patients will experience neurotoxicity (e.g., ICANS) in response to CART immunotherapy, prior to initiation of treatment.

One aspect of the present disclosure provides for a method of determining whether a subject is at risk for developing an immunotherapy-associated neurotoxicity. The method generally comprises obtaining a biological sample from a subject and detecting or measuring a neurofilament light chain (NfL) level in the biological sample. If the subject has an elevated NfL level (e.g., more than about 44 pg/mL), the subject is determined to be at higher risk for developing neurotoxicity; if the subject has a normal level of NfL (e.g., less than about 44 pg/mL), the subject is determined to be at lower risk for developing neurotoxicity.

Another aspect of the present disclosure provides for a method of treating a subject for an immunotherapy-associated neurotoxicity. The method generally comprises obtaining a biological sample from the subject, detecting or measuring a neurofilament light chain (NfL) level in the biological sample, and administering a treatment for the immunotherapy-associated neurotoxicity if the subject has an elevated NfL level (e.g., more than about 44 pg/mL).

As described herein, detecting neurofilament light chain (NfL) in a biological sample (e.g., plasma) can be used as a predictive biomarker for neurotoxicities associated with immunotherapies, such as chimeric antigen receptor-modified (CAR) T cell therapy. It is believed that the use of neurofilament light chain (NfL) levels for predicting which patients will develop drug toxicity associated with a new generation cancer immunotherapies is novel.

Immunotherapies are often associated with toxicity, with many patients developing severe neurological complications. Neurological complications that result from CAR T cell therapy, specifically, have been termed immune effector cell-associated neurotoxicity syndrome (ICANS). Symptoms of ICANS can range from mild confusion to seizures, multifocal strokes, diffuse brain swelling (cerebral edema) and death. Currently, there is no defined treatment for ICANS, and it is believed that there is no method available to delineate who will develop ICANS or other forms of immunotherapy-associated neurotoxicity.

As described in Example 1, individuals who ultimately develop ICANS have early (immediately prior to CART infusion) elevations in plasma NfL levels at levels greater than 44 pg/mL as measured using the Simoa HD-1/HD-X kit. Elevations in plasma NfL levels are further sustained after CAR T infusion. These observations suggest that the risk of developing ICANS reflects pre-CAR T infusion host-factors such as plasma NfL, permitting the use of this unique plasma biomarker to aid in predicting which CAR T recipients are at greatest risk of ICANS. Identification of those patients who will ultimately develop ICANS after CAR T therapy will allow for early prophylactic mitigation approaches. This includes early aggressive interventions or considering alternative therapies to CAR T therapy resulting in improved patient outcomes.

The simple blood-based test described herein successfully identifies which patients are most at risk of developing a severe, potentially life-threatening side effect of a new generation of cancer immunotherapy. It would potentially become standard of care as part of all pre-infusion screening as well as at repeated intervals after infusion. This discovery may also apply to those patients receiving other immune based therapies with the possibility of both severe cytokine release syndrome (CRS) and ICANS, such as CAR T, universal allogeneic CAR T, NK-CAR, or bispecific antibody therapy.

Neurofilament Light Chain (NFL) Detection Methods

Described herein are methods of detecting and quantifying neurofilament light chain (NfL) levels as a predictive biomarker for neurotoxicities associated with immunotherapies such as chimeric antigen receptor-modified (CAR) T cell therapy.

Neurofilaments are structural proteins that form part of the axon of neurons, the fundamental building blocks of the brain and nervous system. They are grouped by their size (i.e., molecular weight), with neurofilament light chain (NfL) being the most extensively studied as a biomarker for neural health. Axonal injury and neuronal death result in leakage and elevation of NfL levels in the cerebral spinal fluid and the blood.

Elevation in plasma NfL is currently being explored as a biomarker for discrimination in multiple neurodegenerative diseases. For example, it has been used to distinguish amyotrophic lateral sclerosis (ALS; also known as Lou Gehrig's disease) from ALS disease-mimics, which carry significantly less morbidity and mortality. It is also being studied as a predictive marker for overall survival in ALS, with higher NfL levels associated with accelerated disease progression and worse clinical prognosis. Plasma and serum NfL levels are also being examined as a biomarker for evaluating disease activity in multiple sclerosis (MS). These results may influence treatment frequency and selection for MS patients in the near future.

Provided herein are methods of detecting NfL levels in a biological sample. Any biological sample containing NfL is suitable. Suitable biological samples may include, but are not limited to blood, serum, plasma, or cerebrospinal fluid (CSF). In a specific embodiment, the biological sample is serum or plasma. In a specific embodiment, the biological sample is serum.

As described herein, NfL levels can be detected in a biological sample using an immunoassay, such as an enzyme-linked immunoassay (ELISA), Single Molecule Array (SiMoA), electrochemiluminescence (ECL), electrochemiluminescence immunoassay (ECLIA)). The biological sample can be exposed to capture antibody designed to specifically bind NfL as an antigen. The biological sample can then be exposed to a secondary antibody conjugated to a label, such as a fluorophore. Binding of the secondary antibody to the capture antibody, followed by detection of the label, allows for the quantitative detection of NfL.

Commercially available immunoassays can be used to detect NfL levels in a biological sample. For example, the Simoa NF-Light™ (HD-1/HD-X) kit (Quanterix™) can be used. The Simoa NF-Light™ kit contains paramagnetic particles coupled with NfL-specific antibodies and detection antibodies capable of generating fluorescent product, which are added sequentially to the biological sample. The fluorescent product and associated quantities of NfL can then be detected using the Simoa HD-X Analyzer, a fully automated bead-based immunoassay platform. Detection of plasma neurofilament light chain can be according to methods described in Simoa® NF-Light™ Advantage Kit HD-1/HD-X Data Sheet or Thebault et al., Front. Neurosci., 25 Mar. 2021.

Once NfL levels are quantified in a biological sample, the NfL levels can be compared to a threshold value that stratifies risk of the subject for developing immunotherapy-associated neurotoxicity or the need for treatment of an immunotherapy-associated neurotoxicity. For example, if the NfL levels in the biological sample are elevated (e.g., higher than the threshold value or higher than a control sample or control population), the subject is determined to be at higher risk for developing immunotherapy-associated neurotoxicity or in need of treatment for immunotherapy-associated neurotoxicity; likewise, if the NfL levels are normal (e.g., lower than the threshold value or the same or lower as a control sample or control population), then the subject is determined to be at lower risk for developing immunotherapy-associated neurotoxicity or not in need of treatment for immunotherapy-associated neurotoxicity. In some embodiments, the threshold value is about 44 pg/mL.

If a subject is determined to be in need of treatment for immunotherapy-associated neurotoxicity, based on NfL levels in a biological sample obtained from the subject, the treatment can be administered to the subject before (e.g., prophylactic), during, or after the subject receives an immunotherapy or before (e.g., prophylactic), during, or after the subject has symptoms associated with immunotherapy associated neurotoxicity. Exemplary treatments include but are not limited to steroids, such as glucocorticoids (e.g., dexamethasone, methylprednisolone), immunosuppressants (e.g., tocilizumab), or antiepileptic drugs (e.g., levetiracetam, phenobarbital, and/or lacosamide).

As described herein, the disclosed methods of obtaining a biological sample from a subject and detecting NfL levels can be performed before, after, or concurrently with the subject receiving immunotherapy.

In some embodiments, a biological sample is obtained from a subject and NfL levels detected in the biological sample less than about six months and more than about 24 hours prior to the subject receiving an immunotherapy. For example, a biological sample can be obtained from a subject and NfL levels detected in the biological sample at about six months or less, about five months or less, about four months or less, about three months or less, about two months or less, about one month or less, about three weeks or less, about two weeks or less, about one week or less, about 96 hours or less, about 72 hours or less, about 48 hours or less, or about 24 hours or more prior to the subject receiving an immunotherapy.

In some embodiments, a biological sample is obtained from a subject and NfL levels are detected in the biological sample less than about 30 days and more than about 1 day after the subject has received an immunotherapy. For example, a biological sample can be obtained from a subject and NfL levels detected in the biological sample at about 30 days or less, about 29 days or less, about 28 days or less, about 27 days or less, about 26 days or less, about 25 days or less, about 24 days or less, about 23 days or less, about 22 days or less, about 21 days or less, about 20 days or less, about 19 days or less, about 18 days or less, about 17 days or less, about 16 days or less, about 15 days or less, about 14 days or less, about 13 days or less about 12 days or less, about 11 days or less, about 10 days or less, about 9 days or less, about 8 days or less, about 7 days or less, about 6 days or less, about 5 days or less, about 4 days or less, about 3 days or less, about 2 days or less, or about 1 day or more after the subject has received an immunotherapy.

In some embodiments, a biological sample is obtained from a subject who will, has, or is receiving an immunotherapy and NfL levels are detected in the biological sample at multiple time points. For example, a biological sample can be obtained from a subject and NfL levels are detected in the biological sample before, during, or after treatment with an immunotherapy. As another example, a biological sample can be obtained from a subject and NfL levels can be detected in the biological sample on multiple days or at multiple time points before and/or after the subject has receiving an immunotherapy.

Immunotherapies

As described herein, the provided methods allow for the identification of subjects who are at risk for developing neurotoxicities in response to immunotherapy or checkpoint immunotherapy. Immunotherapies are a new generation of cancer therapy that has revolutionized the treatment of otherwise terminal cancers, often achieving durable, sustained remission in cancers that were otherwise thought to be refractory to standard first- and second-line therapies. Thousands of patients annually are now treated with these life-saving therapies.

In the context of cancer treatment, immunotherapeutics, generally, rely on the use of immune effector cells and molecules to target and destroy cancer cells. Trastuzumab (Herceptin™) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells. The combination of therapeutic modalities, i.e., direct cytotoxic activity and inhibition or reduction of ErbB2 would provide therapeutic benefit in the treatment of ErbB2 overexpressing cancers.

Examples of immunotherapy can be immune effector cell (IEC) therapy (e.g., CAR T, mesenchymal stem cells) or T cell engaging therapy (e.g., CD19-specific T cell engager, such as blinatumomab, T cell engaging monoclonal antibody, bispecific T cell engager (BiTE) therapy).

In some embodiments, the provided methods are used before, after, or in concurrence with any form of BsMAb therapy. For example, the BsMAb therapy can be any one or more of the currently FDA-approved BsMAb therapies, such as blinatumomab, emicizumab, or amivantamab.

In one aspect of immunotherapy, the tumor cell must bear some marker that is amenable to targeting, i.e., is not present on the majority of other cells. Many tumor markers exist and any of these may be suitable for targeting in the context of the present disclosure. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155. An alternative aspect of immunotherapy is to combine anticancer effects with immune stimulatory effects. Immune stimulating molecules also exist including: cytokines such as IL-2, IL-4, IL-12, GM-CSF, γ-IFN, chemokines such as MIP-1, MCP-1, IL-8 and growth factors such as FLT3 ligand. Combining immune stimulating molecules, either as proteins or using gene delivery in combination with a tumor suppressor has been shown to enhance anti-tumor effects (Ju et al., 2000). Moreover, antibodies against any of these compounds may be used to target the anti-cancer agents discussed herein.

Examples of immunotherapies currently under investigation or in use are immune adjuvants e.g., Mycobacterium bovis, Plasmodium falciparum, dinitrochlorobenzene and aromatic compounds (U.S. Pat. Nos. 5,801,005 and 5,739,169; Hui and Hashimoto, 1998; Christodoulides, et al., 1998), cytokine therapy, e.g., interferons α, β, and γ; IL-1, GM-CSF, TNF (Bukowski, et al., 1998; Davidson, et al., 1998; Hellstrand, et al., 1998) gene therapy, e.g., TNF, IL-1, IL-2, p53 (Qin et al., 1998; Austin-Ward and Villaseca, 1998; U.S. Pat. Nos. 5,830,880 and 5,846,945), and monoclonal antibodies, e.g., anti-ganglioside GM2, anti-HER-2, anti-p185 (Pietras, et al., 1998; Hanibuchi, et al., 1998; U.S. Pat. No. 5,824,311). It is contemplated that one or more anti-cancer therapies may be employed with the gene silencing therapies described herein.

In active immunotherapy, an antigenic peptide, polypeptide or protein, or an autologous or allogenic tumor cell composition or “vaccine” is administered, generally with a distinct bacterial adjuvant (Ravindranath and Morton, 1991; Morton, et al., 1992; Mitchell, et al., 1990; Mitchell, et al., 1993).

In adoptive immunotherapy, the patient's circulating lymphocytes, or tumor infiltrated lymphocytes, are isolated in vitro, activated by lymphokines such as IL-2 or transduced with genes for tumor necrosis, and readministered (Rosenberg, et al., 1988; 1989).

CAR T

In some embodiments, the immunotherapy in accordance with the present disclosure is CART cell therapy (e.g., CD19-specific chimeric antigen receptor T (CAR-T)). Generally, CAR T cell therapy refers to any type of immunotherapy in which a subject's T cells are genetically modified to express chimeric antigen receptors. These chimeric antigen receptors allow the T cells to more effectively recognize and subsequently destroy cancer cells. Typically, T cells are first harvested from a subject, genetically altered to express a CAR targeting an antigen of interest (e.g., an antigen expressed on the surface of a tumor or cancer cell), and then infused back into the subject. Once infused into the subject, CAR T cells bind to the target antigen and are activated, allowing them to proliferate and become cytotoxic.

In some embodiments, the provided methods are used before, after, or in concurrence with any form of CAR T cell therapy. For example, the CAR T cell therapy can be any one or more of the currently FDA-approved CAR T cell therapies, which include anti-CD19 and anti-BCMA CAR T cells such as tisagenlecleucel (Kymriah), axicabtagene ciloleucel (Yescarta), brexucabtagene autoleucel (Tecartus), lisocabtagene maraleucel (Breyanzi), or idecabtagene vicleucel (Abecma). In some embodiments, the CAR T cell therapy is tisagenlecleucel or axicabtagene ciloleucel.

The CAR T cell therapy can also be a non-FDA-approved or experimental CAR T cell therapy (e.g., a CAR T cell therapy undergoing clinical trials), such as anti-CD22, anti-CAIX, anti-PSMA, anti-MUC1, anti-FRα, anti-meso-RNA, anti-CEA, anti-IL13Rα2, anti-HER2, or universal allogenic CART cells.

Checkpoint Immunotherapy

An important function of the immune system is its ability to tell between normal cells in the body and those it sees as “foreign.” This lets the immune system attack the foreign cells while leaving the normal cells alone. To do this, it uses “checkpoints.” Immune checkpoints are molecules on certain immune cells that need to be activated (or inactivated) to start an immune response.

Cancer cells can find ways to use these checkpoints to avoid being attacked by the immune system. But drugs that target these checkpoints hold a lot of promise as a cancer treatment. These drugs are called checkpoint inhibitors. Checkpoint inhibitors used to treat cancer don't work directly on the tumor at all. They only take the brakes off an immune response that has begun but hasn't yet been working at its full force.

Checkpoint immunotherapy has been extensively shown to unleash T cell effector functions to control tumors in many cancer patients. However, tumor cells can evade immunological elimination by recruiting myeloid cells that induce an immunosuppressive state. Recent high dimensional profiling studies have shown that tumor-infiltrating myeloid cells are considerably heterogeneous, and may include both immunostimulatory and immunosuppressive subsets, although they do not fit the M1/M2 paradigm. Thus, depletion of suppressive myeloid cells from tumors, blockade of their functions, or induction of myeloid cells with immunostimulatory properties may provide important approaches for improving immunotherapy strategies, perhaps in synergy with checkpoint blockade.

Any immune checkpoint inhibitor known in the art can be used. For example, a PD-1 inhibitor can be used. These drugs are typically administered IV (intravenously). PD-1 is a checkpoint protein on immune cells called T cells. It normally acts as a type of “off switch” that helps keep the T cells from attacking other cells in the body. It does this when it attaches to PD-L1, a protein on some normal (and cancer) cells. When PD-1 binds to PD-L1, it tells the T cell to leave the other cell alone. Some cancer cells have large amounts of PD-L1, which helps them hide from an immune attack.

Monoclonal antibodies that target either PD-1 or PD-L1 can block this binding and boost the immune response against cancer cells. These drugs have shown a great deal of promise in treating certain cancers.

Examples of drugs that target PD-1 can include: Pembrolizumab (Keytruda), Nivolumab (Opdivo), or Cemiplimab (Libtayo). These drugs have been shown to be helpful in treating several types of cancer, and new cancer types are being added as more studies show these drugs to be effective.

As another example, a PD-L1 inhibitor can be used. Examples of drugs that target PD-L1 can include: Atezolizumab (Tecentriq), Avelumab (Bavencio), or Durvalumab (Imfinzi). These drugs have also been shown to be helpful in treating different types of cancer, and are being studied for use against others.

CTLA-4 is another protein on some T cells that acts as a type of “off switch” to keep the immune system in check. For example, Ipilimumab (Yervoy) is a monoclonal antibody that attaches to CTLA-4 and reduces or blocks its function. This can boost the body's immune response against cancer cells. This drug can be used to treat melanoma of the skin and other cancers.

Bispecific Monoclonal Antibody (BsMAb)

In some embodiments, the immunotherapy in accordance with the present disclosure is bispecific monoclonal antibody (BsMAb) therapy. BsMAbs are synthetic proteins engineered to bind two different antigens simultaneously. For cancer immunotherapies, BsMAbs are typically designed to bind both a cytotoxic cell (e.g., a T cell) and an antigen expressed on a tumor or cancer cell. Engagement of T-cells and activation of antibody-dependent cellular cytotoxicity (ADCC) result in tumor cell death.

In some embodiments, the provided methods are used with, before, after, or in concurrence with any form of BsMAb therapy. For example, the BsMAb therapy can be any one or more of the currently FDA-approved BsMAb therapies, such as blinatumomab, emicizumab, or amivantamab.

Cell Therapy

In cell therapy (also called cellular therapy, cell transplantation, or cytotherapy) viable cells can be injected, grafted, or implanted into a patient in order to effectuate a medicinal effect or therapeutic benefit. For example, transplanting T-cells capable of fighting cancer cells via cell-mediated immunity can be used in the course of immunotherapy.

Stem cell and cell transplantation has gained significant interest by researchers as a potential new therapeutic strategy for a wide range of diseases, in particular for degenerative and immunogenic pathologies.

Allogeneic cell therapy or allogenic transplantation uses donor cells from a different subject than the recipient of the cells. A benefit of an allogenic strategy is that unmatched allogenic cell therapies can form the basis of “off the shelf” products.

Autologous cell therapy or autologous transplantation uses cells that are derived from the subject's own tissues. It could also involve the isolation of matured cells from diseased tissues, to be later re-implanted at the same or neighboring tissues. A benefit of an autologous strategy is that there is limited concern for immunogenic responses or transplant rejection.

Xenogeneic cell therapies or xenotransplantation uses cells from another species. For example, pig derived cells can be transplanted into humans. Xenogeneic cell therapies can involve human cell transplantation into experimental animal models for assessment of efficacy and safety or enable xenogeneic strategies to humans as well.

Immunotherapy-Associated Neurotoxicity

Provided herein are methods allowing for identification of subjects who are at risk for developing neurotoxicities in response to immunotherapy (e.g., immunotherapy-associated neurotoxicity) and methods of treating immunotherapy-associated neurotoxicity.

Immunotherapies have been recently developed for the effective treatment of cancer. However, because immunotherapies modulate the immune system, they can cause a number of immune-related adverse events, including neurotoxicity.

Neurotoxicity refers to any adverse effect on the structure or function of the nervous system occurring after a subject has been exposed to an agent or treatment, such as immunotherapy. For example, neurotoxicity may result in damage to nervous tissue or death of neurons and other cells of the nervous system.

The occurrence of neurotoxicity in a subject after treatment with immunotherapy may be determined if the subject exhibits symptoms associated with neurotoxicity. For example, these symptoms can include aphasia, tremor, dysgraphia, lethargy, obtundation, stupor, coma, limb weakness or numbness, loss of memory or vision, headache, cognitive or behavioral problems, sexual dysfunction, mild confusion, seizures, multifocal strokes, diffuse brain swelling (cerebral edema), intracerebral hemorrhage, or death.

For patients treated with CAR T immunotherapy, between 30-40% are expected to develop neurological complications, such as neurotoxicity in response to treatment, with mild symptoms reported in upwards of 70% of all treated patients; however, many patients can develop more severe neurotoxicities. These neurotoxicities, which can occur days to weeks following CAR T treatment, are collectively termed immune effector cell-associated neurotoxicity syndrome (ICANS).

Initial symptoms of ICANS can include aphasia, dysphasia, impaired fine motor skills, difficulty with word finding, confusion, and somnolence. More severe cases of ICANS may result in motor weakness, seizures, cerebral edema, coma, and even death.

Currently, there is no method available to delineate who will develop ICANS. There is no defined treatment for ICANS, with management instead focusing on aggressive, early supportive care and the use of corticosteroids.

Immune Effector Cell-Associated Neurotoxicity Syndrome (ICANS)

An example of an immunotherapy-associated neurotoxicity can be Immune effector cell-associated neurotoxicity syndrome (ICANS), which is a clinical and neuropsychiatric syndrome that can occur in the days to weeks following administration of certain types of immunotherapy, especially immune effector cell (IEC) and T cell engaging therapies. ICANS has been previously referred to as cytokine release encephalopathy syndrome (CRES) or chimeric antigen receptor T (CAR-T) cell-related encephalopathy or neurotoxicity.

ICANS is seen at varying degrees in patients treated with CAR-T cell therapies or blinatumomab and has been associated with fatal outcomes in rare cases. Given the rapidly expanding use of IEC therapies, it is imperative for all clinicians involved in the care of treated patients to be familiar with the manifestations and management of ICANS.

CAR-T cell therapies are available for treatment of relapsed and refractory hematologic malignancies and are also under investigation in a range of solid tumors. CD19-targeting CAR-T cell products approved by the US Food and Drug Administration (FDA) include: tisagenlecleucel for relapsed/refractory B cell acute lymphoblastic leukemia and relapsed/refractory large B cell lymphoma; axicabtagene ciloleucel for relapsed/refractory large B cell lymphoma and relapsed/refractory follicular lymphoma; brexucabtagene autoleucel for relapsed/refractory mantle cell lymphoma; lisocabtagene maraleucel for relapsed/refractory large B cell lymphoma; or idecabtagene vicleucel for relapsed/refractory multiple myeloma.

Incidence and Risk Factors

The incidence of ICANS varies depending on the type of therapy being delivered, disease and patient characteristics, and because the definition and recognition of the syndrome has changed over time.

Specific products—Therapies most commonly associated with ICANS are chimeric antigen receptor T (CAR-T) cell therapies and blinatumomab, a CD19/CD3 bispecific T cell engager for B cell acute lymphoblastic leukemia.

CAR-T cell therapies—ICANS of any severity occurs in 20 to 70 percent of patients treated with CAR-T cell therapy. Severe ICANS has been observed in the pivotal studies of all five currently FDA approved products.

Differences in the costimulatory domains of each product affect neurotoxicity risk. Among the available products, rates and severity of ICANS are generally higher with axicabtagene ciloleucel and brexucabtagene autoleucel, which have CD28-containing CAR-T constructs and are associated with more rapid T cell expansion kinetics, effector memory T cell differentiation, and glycolic metabolism, compared with tisagenlecleucel, lisocabtagene maraleucel, and idecabtagene vicleucel, which have a 4-1BB-containing CAR construct that supports more oxidative metabolism with slower T cell expansion kinetics, a central memory T cell phenotype, and potentially longer-term persistence.

Structural differences and manufacturing variability may also affect risk, making comparisons of incidence based purely on transgene structure limited. Across different products, higher CAR-T cell doses and peak CAR-T cell expansion are associated with increased ICANS incidence.

Comparisons of toxicity across early trials is difficult due to differences in grading systems (Common Terminology Criteria for Adverse Events (CTCAE) version 4.03 versus Penn Criteria) as well as use of different management algorithms, with earlier studies intervening at higher grades due to concerns of impacting treatment efficacy. With improved management strategies, there has been a relative reduction in toxicities; however, key product differences remain.

Blinatumomab—Neurologic toxicity occurs in approximately 65 percent of patients treated with blinatumomab. The most common manifestations are headache and tremor. CTCAE grade 3 or higher toxicities (severe, life-threatening, or fatal) have been seen in approximately 13 percent of patients, including encephalopathy, seizures, disturbances of speech and consciousness, confusion, disorientation, and imbalance.

Blinatumomab-associated neurotoxicity is the most common reason for dose interruption; however, neurologic symptoms usually resolve with drug hold and typically decrease with subsequent cycles.

Clinical risk factors—Across different products and studies, clinical risk factors associated with increased risk of ICANS include: younger patient age; preexisting neurologic and medical comorbidities; high disease burden of the underlying malignancy; increased intensity of lymphodepleting therapy and cytopenias; or early and severe cytokine release syndrome (CRS) with high levels of inflammatory cytokines.

Of note, the pivotal studies of all approved CAR-T cell therapies excluded patients with active central nervous system (CNS) disease given concern for increased risk of ICANS. However, subsequent work has demonstrated that CAR-T can be used safely in patients with CNS malignancies with appropriate management, as ICANS is likely a neurologic manifestation of an otherwise systemic process.

Pathophysiology

The pathophysiology of ICANS and the mechanisms underlying many of the symptoms are not fully understood. In general, it is thought that systemic inflammation and high levels of circulating cytokines result in endothelial cell activation and blood-brain barrier (BBB) disruption, which in turn causes an inflammatory cascade within the central nervous system (CNS), subsequent alterations in cortical and subcortical function, and diffuse cerebral edema in some cases. Both ICANS and cytokine release syndrome (CRS) are considered an enhanced or supraphysiologic immune response to immune-modulating therapy that activates or engages T cells and/or other immune effector cells (IECs). CAR-T-related ICANS is commonly associated with, and follows, CRS, which other have suggested is a potential mechanistic link. Clinical correlates of severe ICANS overlap with severe CRS, including elevations in C-reactive protein (CRP), ferritin, and cytopenias.

While there was early speculation that neurotoxicity was antigen specific, since ICANS was observed both with the CD19-specific T cell engager, blinatumomab, and with CD19-specific chimeric antigen receptor T (CAR-T) therapies, subsequent reports have identified ICANS in other, non-CD19-associated applications and diseases.

Endothelial activation and disruption of the BBB have been identified as potential mechanisms. The angiopoietin (ANG) and angiopoietin receptor (TIE) axis is disrupted during an initial inflammatory insult, likely mediated, in part, by tumor necrosis factor alpha (TNF-alpha), interleukin (IL) 6, and IL-1. This results in endothelial activation and microvascular permeability, including BBB breakdown. With increased BBB permeability, patients with severe ICANS also exhibit elevated CNS levels of cytokines, protein, and T cell infiltrates.

In addition to CAR-T activation, recruitment and activation of other immune competent cells have been implicated, including myeloid cells, monocytes, and macrophages. Studies in patients with CAR-T cell-associated neurotoxicity have shown elevations in inflammatory cytokines (e.g., interferon gamma (IFN-gamma), IL-6, IL-8, IL-10, granulocyte colony-stimulating factor (G-CSF), granulocyte-macrophage colony-stimulating factor (GM-CSF), monocyte chemoattractant protein 1 (MCP-1), interferon gamma-induced protein 10 (IP-10)), markers of astrocyte injury (e.g., glial fibrillary acidic protein (GFAP), S100 calcium-binding protein B (S100B)), and neurotoxic substances (e.g., glutamate and quinolinic acid).

Clinical Features

The clinical presentation of ICANS can range from mild alterations in the level of consciousness to varying degrees of neurologic dysfunction, including: encephalopathy with confusion and behavioral changes; visual and auditory hallucinations; language dysfunction, speech alterations, and apraxia; headache, fatigue, and tremors; dysgraphia and other fine motor impairment; clinical or subclinical seizures, including status epilepticus; cerebral edema with coma; or death secondary to malignant cerebral edema.

ICANS most often develops within 3 to 10 days after chimeric antigen receptor T (CAR-T) cell administration, but the timing can vary among CAR-T cell products and disease indications. ICANS usually occurs in the context of cytokine release syndrome (CRS), with neurologic symptoms beginning within two to four days of the onset of CRS. However, CRS is not required for ICANS, and the syndromes can occur at different times.

Initial neurologic symptoms are usually characterized by inattention and language deficits. Clinical symptoms can be rapidly progressive within hours to a few days. Close monitoring during this time is critical. Mildly affected patients may be disoriented but able to communicate, with mild expressive and/or receptive language dysfunction. Worsening signs of encephalopathy include decreased level of consciousness, slowness to respond, and disorientation to time and location. Severely affected patients can have language dysfunction or mutism, experience seizures, and be difficult to arouse (i.e., only responsive to tactile or noxious stimulation).

The acute symptoms of ICANS can be considered reversible and usually resolve within 7 to 10 days of onset with adequate management. Nevertheless, neurotoxicity can be life threatening and/or extended, with some patients requiring prolonged intensive care unit (ICU) monitoring and mechanical ventilation for airway protection and management of elevated intracranial pressure (ICP). In fatal cases, the cause of death has primarily been attributed to malignant cerebral edema.

Laboratories in patients with ICANS often, but not always, show evidence of systemic inflammation due to concomitant CRS.

Blood biomarkers that have been previously associated with higher risk for developing neurotoxicity include high lactate dehydrogenase (LDH) levels (as a marker of disease burden), significant thrombocytopenia (as a marker of bone marrow toxicity in heavily pretreated patients), and rising inflammatory markers such as ferritin, C-reactive protein (CRP), and erythrocyte sedimentation rate (ESR; as evidence of cytokine release and immune activation).

Patients with severe ICANS exhibit high serum levels of proinflammatory cytokines such as interleukin (IL) 2, IL-6, IL-15, interferon gamma (IFN-gamma), and tumor necrosis factor alpha (TNF-alpha). While such biomarkers have so far not been routinely used in clinical practice, CRP and ferritin levels are easily obtained, and rising CRP/ferritin is a clinically helpful biomarker associated with an elevated risk for ICANS, but as shown in FIG. 10 and FIG. 1D, NfL is significantly more predictive.

Previous studies of protein elevation and pleocytosis in cerebrospinal fluid (CSF) in patients with ICANS were nonspecific. CSF may be normal or may show mild protein elevation and pleocytosis.

Most patients with ICANS have an abnormal electroencephalogram (EEG), reflective of some degree of encephalopathy and, less commonly, electrographic seizures. Frontal or diffuse theta-delta slowing is the most commonly observed pattern. Other findings include generalized periodic discharges (GPDs), generalized rhythmic delta activity (GRDA), bilateral periodic discharges (BiPEDs), and frank electrographic seizures and status epilepticus. It is not yet clear whether the degree of ICANS correlates with specific EEG findings.

Most patients with ICANS have normal neuroimaging studies, even in the context of clinically established CRS and ICANS. In severe ICANS complicated by increased ICP, computed tomography (CT) and magnetic resonance imaging (MRI) of the brain may show diffuse white matter changes and sulcal effacement, indicative of diffuse cerebral. Other abnormal findings that have been described infrequently in patients with ICANS include cerebral infarctions, subarachnoid or subdural hemorrhage, and focal or diffuse white matter injury.

Focal vascular abnormalities on CT angiography have been described in a minority of patients, including at least one case of vasospasm suggested by partial resolution on two-week follow-up imaging.

Grading

Grading of ICANS has evolved since early clinical trials of chimeric antigen receptor T (CAR-T) therapy. Guidelines from the American Society for Transplantation and Cellular Therapy (ASTCT) attempt to harmonize earlier scales, including the CAR-T Cell Therapy-Associated Toxicity (CARTOX-10) criteria, and recognize the growing diversity in immune effector cell (IEC) therapies and the potential for neurotoxicity outside of CAR-T applications.

The ASTCT grading scale includes a 10-point encephalopathy assessment, termed the “immune effector cell-associated encephalopathy” (ICE) score, which builds on the previous CARTOX-10 element for assessing receptive aphasia. The ICE score has five components: orientation, naming, following commands, writing, and attention.

Patients are graded according to the most severe symptom attributable to ICANS in five domains: encephalopathy (ICE score), level of consciousness, seizure, motor findings, and elevated intracranial pressure (ICP)/cerebral edema.

For example:
Grade 1 (mild)—A patient with grade 1 ICANS may demonstrate inattentiveness, mild disorientation, and mild expressive and/or receptive language dysfunction but will be able to communicate.
Grade 2 (moderate)—A patient with grade 2 ICANS may have a moderately impaired level of consciousness but is responsive to voice, usually slow to respond, and disoriented to time and location.
Grade 3/4 (severe)—Grade 3/4 ICANS includes patients with more severe and significant language dysfunction or mutism, those who are difficult to arouse (i.e., only responsive to tactile or noxious stimulation), and potentially those with seizures.

Although the ICE assessment is useful for screening adults for encephalopathy, it is not optimized for children. For children age <12 years, or those with developmental delay, the Cornell Assessment of Pediatric Delirium (CAPD) is generally recommended to aid in the overall grading of ICANS. Other domains are the same as in adults: level of consciousness, motor symptoms, seizures, and signs of raised ICP.

Of note, neurotoxicity associated with non-IEC therapies (e.g., bispecific antibodies) is graded using the standard Common Terminology Criteria for Adverse Events (CTCAE) version 5.0, as the ICANS/ICE system only pertains to IEC therapies. IECs can be any cell used to modulate an immune response for therapeutic intent, such as dendritic cells, natural killer cells, T cells, and B cells. This includes genetically engineered CAR-T cells and therapeutic vaccines.

Evaluation and Diagnosis

ICANS is a clinical diagnosis of neurologic toxicity attributed to recent administration of an immune effector cell (IEC) therapy or T cell engaging therapy. It is a diagnosis of exclusion after other potential causes of mental status changes or altered neurologic function have been ruled out. The below evaluation procedures can be performed especially when the patient has been determined to be at higher risk for immunotherapy associated neurotoxicity.

Clinical monitoring—Patients who receive chimeric antigen receptor T (CAR-T) cell therapy and blinatumomab require close monitoring for the development of cytokine release syndrome (CRS) and ICANS.

Decisions to treat patients as inpatients or outpatients should be based on product-specific toxicity profiles, the time course of toxicity, and center-specific infrastructure for safety and monitoring.

In the United States, the US Food and Drug Administration (FDA) has mandated risk evaluation and mitigation strategies (REMS), and all centers providing commercial CAR-T cell products must be authorized prior to treating patients. Some FDA-approved products (such as axicabtagene ciloleucel) require daily clinical evaluations for the first week following CAR-T infusion.

A baseline neurologic examination should be performed prior to administration of CAR-T cells in order to establish a patient-specific neurologic baseline.

Daily clinical assessment for ICANS can include:

Physical examination and review of vital signs. More frequent clinical evaluations and bedside assessments are recommended in patients at higher risk for neurotoxicity, such as in patients with fever and other signs of CRS, preexisting neurologic deficits, and any evidence of change in mental status.

Routine neurologic examination. There should be particular focus on subtle deficits in attention and changes in alertness and language function, as these are usually the earliest signs of ICANS. Family members and the nursing team may have insight into subtle personality changes or other deviations from baseline not readily detected by routine examination.

Bedside funduscopy, when possible. Funduscopy is particularly important in patients with altered mental status and visual changes, to assess for signs of increased intracranial pressure (ICP) and possible cerebral edema.

Immune effector cell-associated encephalopathy (ICE) score. The ICE score can be performed as part of any routine assessment during the at-risk period (typically the 30-day period following CAR-T infusion). This helps to standardize, quantify, and trend a patient's neurologic status.

Laboratory review. Serial monitoring of laboratory tests helps to inform the index of suspicion for CRS and ICANS. ICANS usually presents in a temporal relationship to CRS, and most patients develop neurologic deficits within days after CRS onset. Daily laboratory tests should include complete blood counts, chemistry profile, coagulation panel, and routine inflammatory markers, such as ferritin and C-reactive protein (CRP).

Changes in the neurologic examination should be interpreted in the context of a broad differential diagnosis. Patients receiving CAR-T cell therapy are usually heavily pretreated with chemotherapy and at risk for multiple causes of altered mental status, including medication side effects, infection, new or worsening renal or liver dysfunction, and changes in electrolytes and endocrine function.

Additional testing of patients with suspected ICANS may include the following:

Electroencephalography (EEG)—EEG is an important tool in the evaluation of suspected ICANS. Any patient with unexplained altered mental status should undergo EEG recording to diagnose or rule out subclinical or nonconvulsive seizures that otherwise would not be readily detectable by a clinical bedside examination.

While ICANS does not have a specific EEG signature, EEG is often uniquely helpful to support the diagnosis and degree of encephalopathy and offer clues regarding the potential differential diagnosis (e.g., seizures versus metabolic causes). Long-term EEG monitoring can be helpful in patients with prolonged or fluctuating encephalopathy and mental status changes to guide management and adjustment of antiseizure medications.

Neuroimaging—A noncontrast head computed tomography (CT) is indicated in patients with rapidly worsening mental status or acute focal deficits to rule out cerebral edema or other acute abnormalities (e.g., bleeding) and to guide further management, such as escalating care and need for intensive care unit (ICU) monitoring.

When possible, brain magnetic resonance imaging (MRI) is recommended in patients with worsening ICANS as part of the workup for other etiologies of neurologic dysfunction and to evaluate for cerebral edema and more subtle changes in cerebral white matter.

Lumbar puncture—Cerebrospinal fluid (CSF) examination is indicated when there is suspicion of central nervous system (CNS) infection or neoplastic CNS involvement. Neuroimaging, as well as platelet count and coagulation studies, should be reviewed before lumbar puncture to ensure safety.

Differential diagnosis—The differential diagnosis of ICANS is broad, and evaluation can be challenging. Patients treated with CAR-T cells are often quite ill and deconditioned, are often heavily pretreated, and commonly present with more than one clinical symptom (e.g., mental status changes in combination with fever, low blood counts, and/or electrolyte abnormalities).

The list of alternative diagnoses varies depending on the lead clinical symptom(s) (e.g., encephalopathy versus new focal neurologic deficit), the temporal course (e.g., acute, subacute, fluctuating, progressive), and the duration of the abnormality.

Encephalopathy—In any patient with the acute or subacute onset of encephalopathy, initial considerations include adverse effects from medications (e.g., opioids, other sedating medications), metabolic abnormalities (e.g., renal or liver impairment), endocrine dysfunction (e.g., adrenal insufficiency, thyroid dysfunction), infections (including systemic or CNS infections), seizures (clinical or subclinical), and even psychiatric etiologies.

Medication review and appropriate laboratories will identify most of these culprits. EEG can also be helpful and is often necessary to rule out subclinical seizures.

Focal or rapidly progressive deficits—Additional considerations in patients with focal or rapidly worsening deficits include seizure, intracranial hemorrhage, stroke, and intracranial infection.

A noncontrast head CT will serve to identify acute bleeding or evidence of life-threatening cerebral edema. A brain MRI with and without contrast will have better sensitivity for acute ischemia, white matter injury, leptomeningeal processes, and other alternative CNS etiologies. CSF testing is indicated if there is clinical suspicion for meningitis or encephalitis.

Many other neurologic disorders may present in the days to weeks following CAR-T cell therapy with shared features of ICANS. Repeated examinations and multidisciplinary input are often required to establish the correct diagnosis. As examples:

Tumor progression within the CNS—Tumor progression within the CNS can occur at any time in relation to administration of anticancer therapies. The index of suspicion is typically highest in patients being treated for CNS malignancies or with systemic lymphoma/leukemias with a high rate of spread to the nervous system. When neuroimaging and/or CSF are equivocal, repeated examinations and occasionally biopsy may be necessary.

Fludarabine-associated neurotoxicity—Delayed neurologic complications from prior conventional cytotoxic chemotherapy can be a confounding factor. Specifically, fludarabine-associated neurotoxicity may present with delayed and slowly progressive cognitive decline, visual disturbances, peripheral neuropathy, weakness, ataxia, and even death from 20 to 250 days following drug exposure. MRI may demonstrate areas of restricted diffusion and leukoencephalopathy.

Fludarabine is increasingly used as part of the lymphodepleting regimen prior to CAR-T cell administration, and clinicians should be cautious of fludarabine dosing and fluctuations in creatine clearance.

Reversible posterior leukoencephalopathy syndrome (RPLS)—RPLS can be caused by numerous drugs and chemotherapy agents and can be seen in the context of elevated blood pressure. Clinical symptoms may include headache, mental status changes, visual disturbances, and seizures. Brain MRI classically shows cortical or subcortical, posterior-predominant T2/fluid-attenuated inversion recovery (FLAIR) hyperintensities and diffusion restriction, although central patterns are also seen.

Progressive multifocal leukoencephalopathy (PML)—PML is caused by reactivation of the JC polyomavirus and can be seen after chemotherapy or immunomodulatory therapy, or in other conditions associated with an immunocompromised state. PML manifests as abnormal T2/FLAIR hyperintensity on brain MRI, usually beginning in the subcortical white matter of the parietal or occipital lobes. Cortex, cerebellum, and deep gray structures can also be involved. CSF JC virus testing can be diagnostic.

Management

Management of patients with ICANS or for patients determined to be at high risk for ICANS can require vigilance and close supportive care, and early recognition is paramount. Treatment can be supportive or prophylactic and can comprise primarily of glucocorticoids (e.g., beclomethasone; betamethasone; budesonide; cortisone; dexamethasone; hydrocortisone; methylprednisolone; prednisolone; prednisone; triamcinolone) and antiseizure therapy (e.g., levetiracetam, lacosamide, benzodiazepines, valproic acid). While neurologic deficits are considered transient and are usually reversible with appropriate management, fatal outcomes have been reported secondary to malignant cerebral edema.

Treatment recommendations are based primarily on clinical experience and observational data. Consensus guidelines for patient evaluation and management are not yet established, but in development by expert groups, such as the Society for Immunotherapy of Cancer (SITC).

General considerations—Management of patients with ICANS can be challenging, and neurologic symptoms may develop rapidly. Most patients have progressive cancer despite multiple prior therapies and as a result are often deconditioned and frail, medically complicated, and immunosuppressed.

Daily monitoring—A baseline neurologic examination should be performed prior to administration of immunotherapies such as chimeric antigen receptor T (CAR-T) cell therapy and daily for at least the first week after treatment. Components of the daily assessment can be as described above.

Severity assessment—Management decisions can be informed by ICANS severity. The American Society for Transplantation and Cellular Therapy (ASTCT) grading scale is the preferred tool.

Multidisciplinary care—A multidisciplinary team approach can be used to evaluate clinical and neurologic status, which can rapidly change, to address the differential diagnosis in the context of changing neurologic function, and to decide whether specific treatments such as glucocorticoids or antiseizure medications should be given.

Clinical care setting—The clinical care setting should be reviewed on a daily basis to determine whether a patient can be managed on a regular medical floor or needs to be more closely monitored, such as in the setting of an intensive care unit (ICU).

ICU care is generally advised in patients with progressive mental status changes and impaired responsiveness potentially related to worsening cerebral edema and/or status epilepticus, and in patients with higher-grade (grade 3 or 4) ICANS, so that close monitoring of neurologic, cardiovascular, and respiratory function can be provided.

Glucocorticoids—Glucocorticoids are an important component in the supportive management of patients with ICANS. The optimal timing, dose, and duration are not well established, however, and treatment decisions are often influenced not only by ICANS but also concomitant cytokine release syndrome (CRS).

Based on the potential for rapid decline, glucocorticoids can begin in patients with moderate to severe (grade 2) ICANS. Many of these patients will already be receiving such therapy due to concomitant CRS; for those who are not, or who have been tapered to lower doses, high-dose therapy can be administered.

Certain specific product approvals and risk evaluation and mitigation strategies (REMS) programs, such as for lisocabtagene maraleucel, can utilize a lower threshold for starting glucocorticoids (grade if ICANS begins within 72 hours of infusion. Whether this approach reduces or shortens ICANS severity while preserving disease response rates has not yet been established, and cross-study comparisons are difficult because of differential management of ICANS throughout the evolution of CAR-T development.

Short-term high-dose glucocorticoids are generally well tolerated, although they can worsen agitation and delirium in some patients, which can confound the assessment of ICANS. There can also be concerns that high-dose steroids may dampen the efficacy of immune effector cell (IEC) therapy. However, in the absence of a clear alternative, the risks of worsening ICANS demand treatment, particularly for severely affected patients.

Observational evidence suggests that a brief course of steroids (e.g., about seven days or fewer or between about 1 and about 10 days) may shorten the course of ICANS without negatively affecting long-term cancer outcomes. However, definitive data are not yet available.

Dose and duration—While the specific dose and optimal daily dosing regimen is not yet generally established, most CRS and ICANS protocols suggest the use of dexamethasone at a starting dose of 10 mg every 6 to 12 hours with a plan to taper over the following two to five days (e.g., up to about 10 days). Generally, most patients will show rapid clinical improvement within hours to days of glucocorticoid initiation. Patients who do not show improvement over hours to days should be examined for alternative etiologies.

In the absence of clear life-threatening seizures or cerebral edema, it is favored to begin a taper after two to five days of high-dose steroids, since prolonged steroid use has been associated with inferior outcomes and steroid-related complications, including delirium, infections, and adrenal insufficiency. For example, a typical taper can be a 25 to 50 percent reduction in the total daily dose of steroid every 24 to 48 hours.

Once patients appear to have returned to their previous neurologic baseline, steroids should be tapered completely off in an effort to avoid the potentially negative impact on the anticancer effect of CAR-T cell therapy. In one study, steroid use for less than about 10 days appeared not to influence overall response to CAR-T cell therapy, although longer courses may be associated with worse clinical outcomes.

Depending on the dose and duration of glucocorticoids, infectious prophylaxis may be indicated given an elevated risk of fungal, bacterial, and viral infections.

Refractory edema—Refractory cerebral edema with acutely increasing intracranial pressure (ICP) is a neurologic emergency. In the rare cases in which edema progresses rapidly despite steroids, patients require aggressive osmotic therapies such as mannitol and hypertonic saline in an attempt to lower ICP.

Seizure prophylaxis and management—Patients with ICANS are at increased risk for seizures. It can be difficult or impossible at the bedside to distinguish fluctuating encephalopathy from seizures, however, and electroencephalography (EEG) may take time to obtain.

From a practical standpoint, antiseizure drug therapy in most patients with suspected ICANS can be administered at the time of the initial presentation of neurologic symptoms. Prophylactic therapy is also reasonable to consider in patients deemed at high risk for seizures, such as those with prior seizure history, concerning EEG findings, or neoplastic brain lesions.

Levetiracetam is the preferred antiseizure medication to be administered in this patient population due to limited drug-drug interactions and less concern for added cardio- and hepatic toxicity. The usual starting dose is 500 mg twice daily.

For patients with clinical or electrographic seizures, dose adjustments and other antiseizure medications (e.g., lacosamide, benzodiazepines, valproic acid) may be administered. Interpretation of EEG patterns can be difficult and often requires continuous monitoring with clinical correlation. Patients with frequent seizures or concern for possible nonconvulsive status epilepticus should be managed in an ICU setting.

The duration of antiseizure prophylaxis after recovery from ICANS has not been generally established. Generally, most patients can be tapered off treatment safely within several weeks after CAR-T cell therapy.

Role of Other Therapies

Tocilizumab—For patients with moderate to severe CRS with or without ICANS, the anti-interleukin (IL) 6 receptor tocilizumab is typically given in combination with glucocorticoids. Earlier intervention with tocilizumab in a patient with both CRS and ICANS may possibly decrease the severity of ICANS.

In patients with ICANS who do not have concurrent CRS, there may not be a role for tocilizumab, which poorly crosses the blood-brain barrier (BBB). Prophylactic use of tocilizumab has been previously suspected of potentially worsening ICANS by increasing IL-6 levels in cerebrospinal fluid (CSF).

Investigational therapies—A number of novel approaches to block inflammatory cytokines are under investigation, including blockade of IL-6 with siltuximab, IL-1 with anakinra, and granulocyte-macrophage colony-stimulating factor (GM-CSF) with lenzilumab. However, their effects on CAR-T cell therapy and ICANS are generally unknown.

Long-Term Outcomes

There has been some debate about possible long-term sequelae of ICANS in patients who initially developed neurologic symptoms during the immediate post-chimeric antigen receptor T (CAR-T) cell phase, but then fully recovered and achieved complete remission from their underlying cancer.

In a study of 40 long-term survivors after CAR-T cell therapies, 48 percent of patients reported at least one clinically meaningful negative neuropsychiatric outcome (e.g., anxiety, depression, cognitive difficulty). In the same study, younger age was associated with worse long-term global mental health, anxiety, and depression. Large long-term follow-up studies are needed to better understand the risk of delayed adverse neurologic outcomes following CAR-T therapies.

### SUMMARY

Immune effector cell-associated neurotoxicity syndrome (ICANS) is a neuropsychiatric syndrome that occurs in up to 70 percent of patients following administration of certain types of immunotherapy, especially chimeric antigen receptor T (CAR-T) cell therapy and the T cell engaging monoclonal antibody, blinatumomab. The pathophysiology of ICANS is not well understood. It is generally believed that systemic inflammation and high levels of circulating cytokines lead to endothelial cell activation, blood-brain barrier (BBB) disruption, and an inflammatory central nervous system (CNS) cascade. ICANS usually occurs in the context of cytokine release syndrome (CRS), beginning between about 3 days to about 10 days after CAR-T cell administration and within about 2 days to about 4 days of CRS onset. The most common symptoms are alterations in level of consciousness, confusion, behavioral changes, and speech and language abnormalities. Patients are at increased risk for seizures, diffuse cerebral edema, and elevated intracranial pressure (ICP). ICANS is graded according to the most severe symptom in five domains: encephalopathy (immune effector cell-associated encephalopathy (ICE) score), level of consciousness, seizure, motor findings, and elevated ICP/cerebral edema. ICANS can be suspected in a patient who develops new neurologic symptoms in the setting of recent immune effector cell (IEC) therapy. Currently, it is generally a diagnosis of exclusion after other potential causes of mental status changes or altered neurologic function have been ruled out. Neuroimaging and electroencephalography (EEG) can be required in many patients to evaluate for alternative etiologies and diagnose seizures. Cerebrospinal fluid (CSF) examination is indicated when there is suspicion for CNS infection. Management of ICANS can require vigilance and close supportive care, and early recognition is paramount. Currently, treatment is supportive and consists primarily of glucocorticoids and antiseizure medications. Based on the potential for rapid decline, it is generally recommended to treat all patients with moderate to severe (grade 2) ICANS with glucocorticoids, thus patients at risk for ICANS can also be treated. Many of these patients may already be receiving such therapy due to concomitant CRS; for those who are not, or who have been tapered to lower doses, high-dose therapy is often started. Patients are at risk for seizures, but clinical diagnosis is complicated and often confounded by encephalopathy. It is generally suggested that starting most patients on an antiseizure medication such as levetiracetam at the time of first neurologic symptoms. Intensive care unit (ICU) care is generally advised in patients with progressive mental status changes and impaired responsiveness potentially related to worsening cerebral edema and/or status epilepticus, and in patients with higher-grade (grade 3 or 4) ICANS.

Therapeutic Methods

Also provided is a process of treating, preventing, or reversing immunotherapy-associated immunotoxicity in a subject in need of administration of a therapeutically effective amount of a therapeutic agent for immunotherapy-associated immunotoxicity (e.g., symptomatic, prophylactic, acute/abortive), so as to substantially inhibit immunotherapy-associated immunotoxicity, slow the progress of immunotherapy-associated immunotoxicity, or limit the development of immunotherapy-associated immunotoxicity.

Methods described herein are generally performed on a subject in need thereof. A subject in need of the therapeutic methods described herein can be a subject having, diagnosed with, suspected of having, or at risk for developing immunotherapy-associated immunotoxicity. A determination of the need for treatment will typically be assessed by a history, physical exam, or diagnostic tests consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans or chickens. For example, the subject can be a human subject.

Generally, a safe and effective amount of a therapeutic agent for immunotherapy-associated immunotoxicity is, for example, an amount that would cause the desired therapeutic effect in a subject while minimizing undesired side effects. In various embodiments, an effective amount of a therapeutic agent for immunotherapy-associated immunotoxicity described herein can substantially inhibit immunotherapy-associated immunotoxicity, slow the progress of immunotherapy-associated immunotoxicity, or limit the development of immunotherapy-associated immunotoxicity.

According to the methods described herein, administration can be parenteral, pulmonary, oral, topical, intradermal, intramuscular, intraperitoneal, intravenous, intratumoral, intrathecal, intracranial, intracerebroventricular, subcutaneous, intranasal, epidural, ophthalmic, buccal, or rectal administration.

When used in the treatments described herein, a therapeutically effective amount of a therapeutic agent for immunotherapy-associated immunotoxicity can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to treat or prevent immunotherapy-associated immunotoxicity.

The amount of a composition described herein that can be combined with a pharmaceutically acceptable carrier to produce a single dosage form will vary depending upon the subject or host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; the activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4^{th} ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes preventing, reversing, or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or a physician.

Administration of a therapeutic agent for immunotherapy-associated immunotoxicity can occur as a single event or over a time course of treatment. For example, a therapeutic agent for immunotherapy-associated immunotoxicity can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to or before, concurrent with, or after conventional treatment modalities for cancer or immunotherapy-associated immunotoxicity.

A therapeutic agent for immunotherapy-associated immunotoxicity can be administered simultaneously or sequentially with another agent, such as an immunotherapy, an antibiotic, an anti-inflammatory, or another agent. For example, a therapeutic agent for immunotherapy-associated immunotoxicity can be administered simultaneously with another agent, such as an immunotherapy, an antibiotic, or an anti-inflammatory. Simultaneous administration can occur through administration of separate compositions, each containing one or more of a therapeutic agent for immunotherapy-associated immunotoxicity, an immunotherapy, an antibiotic, an anti-inflammatory, or another agent. Simultaneous administration can occur through administration of one composition containing two or more of a therapeutic agent for immunotherapy-associated immunotoxicity, an immunotherapy, an antibiotic, an anti-inflammatory, or another agent. A therapeutic agent for immunotherapy-associated immunotoxicity can be administered sequentially with an immunotherapy, an antibiotic, an anti-inflammatory, or another agent. For example, a therapeutic agent for immunotherapy-associated immunotoxicity can be administered before or after administration of an immunotherapy, an antibiotic, an anti-inflammatory, or another agent.

Active compounds are administered at a therapeutically effective dosage sufficient to treat a condition associated with a condition in a patient. For example, the efficacy of a compound can be evaluated in an animal model system that may be predictive of efficacy in treating the disease in a human or another animal, such as the model systems shown in the examples and drawings.

An effective dose range of a therapeutic can be extrapolated from effective doses determined in animal studies for a variety of different animals. In general, a human equivalent dose (HED) in mg/kg can be calculated in accordance with the following formula (see e.g., Reagan-Shaw et al., FASEB J., 22(3):659-661, 2008, which is incorporated herein by reference):

HED(mg/kg)=Animal dose(mg/kg)×(Animal Kₘ/Human Kₘ)

Use of the Kₘ factors in conversion results in more accurate HED values, which are based on body surface area (BSA) rather than only on body mass. Kₘ values for humans and various animals are well known. For example, the Kₘ for an average 60 kg human (with a BSA of 1.6 m²) is 37, whereas a 20 kg child (BSA 0.8 m²) would have a Kₘ of 25. Kₘ for some relevant animal models are also well known, including: mice Kₘ of 3 (given a weight of 0.02 kg and BSA of 0.007); hamster Kₘ of 5 (given a weight of 0.08 kg and BSA of 0.02); rat Kₘ of 6 (given a weight of 0.15 kg and BSA of 0.025) and monkey Kₘ of 12 (given a weight of 3 kg and BSA of 0.24).

Precise amounts of the therapeutic composition depend on the judgment of the practitioner and are peculiar to each individual. Nonetheless, a calculated HED dose provides a general guide. Other factors affecting the dose include the physical and clinical state of the patient, the route of administration, the intended goal of treatment, and the potency, stability, and toxicity of the particular therapeutic formulation.

The actual dosage amount of a compound of the present disclosure or composition comprising a compound of the present disclosure administered to a subject may be determined by physical and physiological factors such as type of animal treated, age, sex, body weight, severity of condition, the type of disease being treated, previous or concurrent therapeutic interventions, idiopathy of the subject and on the route of administration. These factors may be determined by a skilled artisan. The practitioner responsible for administration will typically determine the concentration of active ingredient(s) in a composition and appropriate dose(s) for the individual subject. The dosage may be adjusted by the individual physician in the event of any complication.

In some embodiments, the therapeutic agent (e.g., a steroid) for immunotherapy-associated immunotoxicity may be administered in an amount from about 1 mg; about 2 mg; about 3 mg; about 4 mg; about 5 mg; about 6 mg; about 7 mg; about 8 mg; about 9 mg; about 10 mg; about 11 mg; about 12 mg; about 13 mg; about 14 mg; about 15 mg; about 16 mg; about 17 mg; about 18 mg; about 19 mg; about 20 mg; about 21 mg; about 22 mg; about 23 mg; about 24 mg; about 25 mg; about 26 mg; about 27 mg; about 28 mg; about 29 mg; about 30 mg; about 31 mg; about 32 mg; about 33 mg; about 34 mg; about 35 mg; about 36 mg; about 37 mg; about 38 mg; about 39 mg; about 40 mg; about 41 mg; about 42 mg; about 43 mg; about 44 mg; about 45 mg; about 46 mg; about 47 mg; about 48 mg; about 49 mg; about 50 mg; about 51 mg; about 52 mg; about 53 mg; about 54 mg; about 55 mg; about 56 mg; about 57 mg; about 58 mg; about 59 mg; about 60 mg; about 61 mg; about 62 mg; about 63 mg; about 64 mg; about 65 mg; about 66 mg; about 67 mg; about 68 mg; about 69 mg; about 70 mg; about 71 mg; about 72 mg; about 73 mg; about 74 mg; about 75 mg; about 76 mg; about 77 mg; about 78 mg; about 79 mg; about 80 mg; about 81 mg; about 82 mg; about 83 mg; about 84 mg; about 85 mg; about 86 mg; about 87 mg; about 88 mg; about 89 mg; about 90 mg; about 91 mg; about 92 mg; about 93 mg; about 94 mg; about 95 mg; about 96 mg; about 97 mg; about 98 mg; about 99 mg; or about 100 mg.

In some embodiments, the therapeutic agent (e.g., an anti-seizure medication) for immunotherapy-associated immunotoxicity may be administered in an amount from about 1 mg; about 2 mg; about 3 mg; about 4 mg; about 5 mg; about 6 mg; about 7 mg; about 8 mg; about 9 mg; about 10 mg; about 11 mg; about 12 mg; about 13 mg; about 14 mg; about 15 mg; about 16 mg; about 17 mg; about 18 mg; about 19 mg; about 20 mg; about 21 mg; about 22 mg; about 23 mg; about 24 mg; about 25 mg; about 26 mg; about 27 mg; about 28 mg; about 29 mg; about 30 mg; about 31 mg; about 32 mg; about 33 mg; about 34 mg; about 35 mg; about 36 mg; about 37 mg; about 38 mg; about 39 mg; about 40 mg; about 41 mg; about 42 mg; about 43 mg; about 44 mg; about 45 mg; about 46 mg; about 47 mg; about 48 mg; about 49 mg; about 50 mg; about 51 mg; about 52 mg; about 53 mg; about 54 mg; about 55 mg; about 56 mg; about 57 mg; about 58 mg; about 59 mg; about 60 mg; about 61 mg; about 62 mg; about 63 mg; about 64 mg; about 65 mg; about 66 mg; about 67 mg; about 68 mg; about 69 mg; about 70 mg; about 71 mg; about 72 mg; about 73 mg; about 74 mg; about 75 mg; about 76 mg; about 77 mg; about 78 mg; about 79 mg; about 80 mg; about 81 mg; about 82 mg; about 83 mg; about 84 mg; about 85 mg; about 86 mg; about 87 mg; about 88 mg; about 89 mg; about 90 mg; about 91 mg; about 92 mg; about 93 mg; about 94 mg; about 95 mg; about 96 mg; about 97 mg; about 98 mg; about 99 mg; about 100 mg; about 110 mg; about 120 mg; about 130 mg; about 140 mg; about 150 mg; about 160 mg; about 170 mg; about 180 mg; about 190 mg; about 200 mg; about 210 mg; about 220 mg; about 230 mg; about 240 mg; about 250 mg; about 260 mg; about 270 mg; about 280 mg; about 290 mg; about 300 mg; about 310 mg; about 320 mg; about 330 mg; about 340 mg; about 350 mg; about 360 mg; about 370 mg; about 380 mg; about 390 mg; about 400 mg; about 410 mg; about 420 mg; about 430 mg; about 440 mg; about 450 mg; about 460 mg; about 470 mg; about 480 mg; about 490 mg; about 500 mg; about 510 mg; about 520 mg; about 530 mg; about 540 mg; about 550 mg; about 560 mg; about 570 mg; about 580 mg; about 590 mg; about 600 mg; about 610 mg; about 620 mg; about 630 mg; about 640 mg; about 650 mg; about 660 mg; about 670 mg; about 680 mg; about 690 mg; about 700 mg; about 710 mg; about 720 mg; about 730 mg; about 740 mg; about 750 mg; about 760 mg; about 770 mg; about 780 mg; about 790 mg; about 800 mg; about 810 mg; about 820 mg; about 830 mg; about 840 mg; about 850 mg; about 860 mg; about 870 mg; about 880 mg; about 890 mg; about 900 mg; about 910 mg; about 920 mg; about 930 mg; about 940 mg; about 950 mg; about 960 mg; about 970 mg; about 980 mg; about 990 mg; or about 1000 mg.

In some embodiments, the therapeutic agent for immunotherapy-associated immunotoxicity may be administered in an amount from about 1 mg/kg to about 100 mg/kg, or about 1 mg/kg to about 50 mg/kg, or about 1 mg/kg to about 25 mg/kg, or about 1 mg/kg to about 15 mg/kg, or about 1 mg/kg to about 10 mg/kg, or about 1 mg/kg to about 5 mg/kg, or about 3 mg/kg. In some embodiments, a therapeutic agent can be administered in a range of about 1 mg/kg to about 200 mg/kg, or about 50 mg/kg to about 200 mg/kg, or about 50 mg/kg to about 100 mg/kg, or about 75 mg/kg to about 100 mg/kg, or about 100 mg/kg.

The effective amount may be less than 1 mg/kg/day, less than 500 mg/kg/day, less than 250 mg/kg/day, less than 100 mg/kg/day, less than 50 mg/kg/day, less than 25 mg/kg/day or less than 10 mg/kg/day. It may alternatively be in the range of 1 mg/kg/day to 200 mg/kg/day.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal.

Agents and compositions described herein can be administered in a variety of methods well known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency; improve taste of the product; or improve shelf life of the product.

Cancer

Methods and compositions as described herein can be used for determining a subject's risk for neurotoxicity in response to treatment. For example, the subject can have or being treated for Acute Lymphoblastic Leukemia (ALL); Acute Myeloid Leukemia (AML); Adrenocortical Carcinoma; AIDS-Related Cancers; Kaposi Sarcoma (Soft Tissue Sarcoma); AIDS-Related Lymphoma (Lymphoma); Primary CNS Lymphoma (Lymphoma); Anal Cancer; Appendix Cancer; Gastrointestinal Carcinoid Tumors; Astrocytomas; Atypical Teratoid/Rhabdoid Tumor, Childhood, Central Nervous System (Brain Cancer); Basal Cell Carcinoma of the Skin; Bile Duct Cancer; Bladder Cancer; Bone Cancer (including Ewing Sarcoma and Osteosarcoma and Malignant Fibrous Histiocytoma); Brain Tumors; Breast Cancer; Bronchial Tumors; Burkitt Lymphoma; Carcinoid Tumor (Gastrointestinal); Childhood Carcinoid Tumors; Cardiac (Heart) Tumors; Central Nervous System cancer; Atypical Teratoid/Rhabdoid Tumor, Childhood (Brain Cancer); Embryonal Tumors, Childhood (Brain Cancer); Germ Cell Tumor, Childhood (Brain Cancer); Primary CNS Lymphoma; Cervical Cancer; Cholangiocarcinoma; Bile Duct Cancer Chordoma; Chronic Lymphocytic Leukemia (CLL); Chronic Myelogenous Leukemia (CML); Chronic Myeloproliferative Neoplasms; Colorectal Cancer; Craniopharyngioma (Brain Cancer); Cutaneous T-Cell; Ductal Carcinoma In Situ (DCIS); Embryonal Tumors, Central Nervous System, Childhood (Brain Cancer); Endometrial Cancer (Uterine Cancer); Ependymoma, Childhood (Brain Cancer); Esophageal Cancer; Esthesioneuroblastoma; Ewing Sarcoma (Bone Cancer); Extracranial Germ Cell Tumor; Extragonadal Germ Cell Tumor; Eye Cancer; Intraocular Melanoma; Intraocular Melanoma; Retinoblastoma; Fallopian Tube Cancer; Fibrous Histiocytoma of Bone, Malignant, or Osteosarcoma; Gallbladder Cancer; Gastric (Stomach) Cancer; Gastrointestinal Carcinoid Tumor; Gastrointestinal Stromal Tumors (GIST) (Soft Tissue Sarcoma); Germ Cell Tumors; Central Nervous System Germ Cell Tumors (Brain Cancer); Childhood Extracranial Germ Cell Tumors; Extragonadal Germ Cell Tumors; Ovarian Germ Cell Tumors; Testicular Cancer; Gestational Trophoblastic Disease; Hairy Cell Leukemia; Head and Neck Cancer; Heart Tumors; Hepatocellular (Liver) Cancer; Histiocytosis, Langerhans Cell; Hodgkin Lymphoma; Hypopharyngeal Cancer; Intraocular Melanoma; Islet Cell Tumors; Pancreatic Neuroendocrine Tumors; Kaposi Sarcoma (Soft Tissue Sarcoma); Kidney (Renal Cell) Cancer; Langerhans Cell Histiocytosis; Laryngeal Cancer; Leukemia; Lip and Oral Cavity Cancer; Liver Cancer; Lung Cancer (Non-Small Cell and Small Cell); Lymphoma; Male Breast Cancer; Malignant Fibrous Histiocytoma of Bone or Osteosarcoma; Melanoma; Melanoma, Intraocular (Eye); Merkel Cell Carcinoma (Skin Cancer); Mesothelioma, Malignant; Metastatic Cancer; Metastatic Squamous Neck Cancer with Occult Primary; Midline Tract Carcinoma Involving NUT Gene; Mouth Cancer; Multiple Endocrine Neoplasia Syndromes; Multiple Myeloma/Plasma Cell Neoplasms; Mycosis Fungoides (Lymphoma); Myelodysplastic Syndromes, Myelodysplastic/Myeloproliferative Neoplasms; Myelogenous Leukemia, Chronic (CML); Myeloid Leukemia, Acute (AML); Myeloproliferative Neoplasms; Nasal Cavity and Paranasal Sinus Cancer; Nasopharyngeal Cancer; Neuroblastoma; Non-Hodgkin Lymphoma; Non-Small Cell Lung Cancer; Oral Cancer, Lip or Oral Cavity Cancer; Oropharyngeal Cancer; Osteosarcoma and Malignant Fibrous Histiocytoma of Bone; Ovarian Cancer Pancreatic Cancer; Pancreatic Neuroendocrine Tumors (Islet Cell Tumors); Papillomatosis; Paraganglioma; Paranasal Sinus and Nasal Cavity Cancer; Parathyroid Cancer; Penile Cancer; Pharyngeal Cancer; Pheochromocytoma; Pituitary Tumor; Plasma Cell Neoplasm/Multiple Myeloma; Pleuropulmonary Blastoma; Pregnancy and Breast Cancer; Primary Central Nervous System (CNS) Lymphoma; Primary Peritoneal Cancer; Prostate Cancer; Rectal Cancer; Recurrent Cancer Renal Cell (Kidney) Cancer; Retinoblastoma; Rhabdomyosarcoma, Childhood (Soft Tissue Sarcoma); Salivary Gland Cancer; Sarcoma; Childhood Rhabdomyosarcoma (Soft Tissue Sarcoma); Childhood Vascular Tumors (Soft Tissue Sarcoma); Ewing Sarcoma (Bone Cancer); Kaposi Sarcoma (Soft Tissue Sarcoma); Osteosarcoma (Bone Cancer); Uterine Sarcoma; Sezary Syndrome (Lymphoma); Skin Cancer; Small Cell Lung Cancer; Small Intestine Cancer; Soft Tissue Sarcoma; Squamous Cell Carcinoma of the Skin; Squamous Neck Cancer with Occult Primary, Metastatic; Stomach (Gastric) Cancer; T-Cell Lymphoma, Cutaneous; Lymphoma; Mycosis Fungoides and Sezary Syndrome; Testicular Cancer; Throat Cancer; Nasopharyngeal Cancer; Oropharyngeal Cancer; Hypopharyngeal Cancer; Thymoma and Thymic Carcinoma; Thyroid Cancer; Thyroid Tumors; Transitional Cell Cancer of the Renal Pelvis and Ureter (Kidney (Renal Cell) Cancer); Ureter and Renal Pelvis; Transitional Cell Cancer (Kidney (Renal Cell) Cancer; Urethral Cancer; Uterine Cancer, Endometrial; Uterine Sarcoma; Vaginal Cancer; Vascular Tumors (Soft Tissue Sarcoma); Vulvar Cancer; or Wilms Tumor. Brain or spinal cord tumors can be acoustic neuroma; astrocytoma, atypical teratoid rhabdoid tumor (ATRT); brain stem glioma; chordoma; chondrosarcoma; choroid plexus; CNS lymphoma; craniopharyngioma; cysts; ependymoma; ganglioglioma; germ cell tumor; glioblastoma (GBM); glioma, hemangioma; juvenile pilocytic astrocytoma (JPA); lipoma; lymphoma; medulloblastoma; meningioma; metastatic brain tumor; neurilemmomas; neurofibroma; neuronal & mixed neuronal-glial tumors; non-Hodgkin lymphoma; oligoastrocytoma; oligodendroglioma; optic nerve glioma, pineal tumor; pituitary tumor; primitive neuroectodermal (PNET); rhabdoid tumor; or schwannoma. An astrocytoma can be grade I pilocytic astrocytoma, grade II—low-grade astrocytoma, grade III anaplastic astrocytoma, or grade IV glioblastoma (GBM), or a juvenile pilocytic astrocytoma. A glioma can be a brain stem glioma, ependymoma, mixed glioma, optic nerve glioma, or subependymoma.

A control sample or a reference sample as described herein can be a sample from a healthy subject. A reference value can be used in place of a control or reference sample, which was previously obtained from a healthy subject or a group of healthy subjects. A control sample or a reference sample can also be a sample with a known amount of a detectable compound or a spiked sample.

Compositions and methods described herein utilizing molecular biology protocols can be according to a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1: Pre-Infusion Neurofilament Light Chain (NFL) Levels Predict the Development of Immune Effector Cell-Associated Neurotoxicity Syndrome (ICANS)

This example describes predicting which patients will develop drug toxicity associated with immunotherapy, such as CAR T cell therapy.

### BACKGROUND

Chimeric antigen receptor-modified (CAR) T cell therapy has revolutionized the treatment of refractory B cell malignancies. Neurological side effects are common, with symptoms observed in approximately half of all patients. Termed immune effector cell-associated neurotoxicity syndrome (ICANS), symptoms range from mild encephalopathy to seizures, and diffuse cerebral edema. There remains a critical need to identify patients most at risk for ICANS. Yet a biomarker for the development of ICANS is lacking. Our two-center study evaluated pre-infusion levels of plasma neurofilament light chain (NfL), a marker of neurodegeneration, as a predictive biomarker the development of ICANS.

Methods

Study inclusion criteria included available pre-infusion (up to 4 weeks prior to lymphodepletion) plasma from patients treated with a CAR T cell therapy (n=30, 36% with ICANS, ASTCT consensus ICANS grade range 1-4). Exclusion criteria included confounding diagnoses known to elevate NfL levels (e.g. dementia, recent stroke). Plasma NfL was assayed using a Simoa HD-X kit (Quanterix™). Demographic (age, sex), oncologic (primary, stage, mean tumor volume (MTV), and history of central nervous system (CNS) involvement), and medical history (history of non-oncologic CNS disease or neuropathy) were obtained from the medical record. MTV was derived from total lesion burden on pre-infusion positron emission tomography (PET) scans using a 41% maximum standard uptake value (SUV) threshold. Pre-infusion (i.e. during lymphodepletion) and Post-infusion Day 1 (D1) platelet count, C-reactive protein (CRP), fibrinogen, lactate dehydrogenase (LDH), and ferritin levels were also obtained from the medical record. Group comparisons used log-rank testing, followed by receiver operating characteristic (ROC) curve classification and hierarchical clustering. Validation testing used a 10,000 permutation testing on 80% of the data. Finally, demographic and clinical characteristics were correlated with pre-infusion biomarkers using point-biserial and Spearman (rank) correlation.

Results

Our results demonstrated that individuals who would go on to develop ICANS had elevations in pre-infusion NfL ([87.6 v 29.4 pg/ml, FIG. 1A], p=0.00004) with excellent classification accuracy for the development of ICANS (AUC 0.96; FIG. 1B), sensitivity (0.91) and specificity (0.95). NfL further correlated with ICANS development (r=0.74, p<0.0001; FIG. 1C). Among known post-infusion risk factors, D1 ferritin had the highest classification accuracy, but was inferior to baseline NfL (p<0.05; FIG. 1B). Both baseline NfL and D1 ferritin elevations clustered with ICANS grade (FIG. 1D).

### CONCLUSION

Our findings show that pre-infusion plasma NfL levels are a robust early marker for the development of ICANS that exceeds known post-infusion markers. This suggests the risk of developing ICANS reflects pre-existing latent neuroaxonal injury. Predictive identification of patients at risk of developing ICANS prior to cellular infusion would permit early, preemptive or prophylactic ICANS-directed therapies, thereby improving patient outcomes.

1-35. (canceled)

36. A method of determining a risk for developing an immunotherapy-associated neurotoxicity for a subject in need thereof, the method comprising:
providing a biological sample from the subject, wherein the biological sample is obtained up to 30 days before the subject is expected to receive an immunotherapy;
measuring a neurofilament light chain (NfL) level in the biological sample; and
determining the risk for the subject, wherein:
the subject is determined have a high risk for developing the immunotherapy-associated neurotoxicity if the subject has an elevated NfL level compared to a control or if the subject has an NfL level of more than about 44 pg/mL, or
the subject is determined to have a normal or low risk for developing the immunotherapy-associated neurotoxicity if the subject does not have an elevated level of NfL compared to a control or if the subject has an NfL level of less than about 44 pg/mL.

37. The method of claim 36, wherein the biological sample comprises serum, plasma, or cerebrospinal fluid (CSF).

38. The method of claim 36, wherein the immunotherapy-associated neurotoxicity is immune effector cell-associated neurotoxicity syndrome (ICANS).

39. The method of claim 36, wherein the immunotherapy comprises an immune effector cell therapy.

40. The method of claim 39, wherein the immune effector cell therapy is a CAR T cell therapy selected from engineered CAR T, universal allogeneic CAR T, CD19-specific CAR T, anti-CD19 CAR T, anti-BCMA CAR T, anti-CD22, anti-CAIX, anti-PSMA, anti-MUC1, anti-FRα, anti-meso-RNA, anti-CEA, anti-IL13Rα2, or anti-HER2.

41. The method of claim 36, wherein the immunotherapy comprises a bispecific antibody therapy.

42. A method of treating immunotherapy-associated neurotoxicity in a subject in need thereof, the method comprising:
providing a biological sample from the subject, wherein the subject has an immunotherapy-associated neurotoxicity;
measuring a neurofilament light chain (NfL) level in the biological sample; and
administering a treatment for the immunotherapy-associated neurotoxicity before, after, and/or during an immunotherapy when the subject has an elevated NfL level compared to a control or when the subject has an NfL level of more than about 44 pg/mL.

43. The method of claim 42, wherein the biological sample comprises serum, plasma, or cerebrospinal fluid (CSF).

44. The method of claim 42, wherein measuring the NfL level in the biological sample is performed up to 30 days before the subject is expected to receive the immunotherapy.

45. The method of claim 42, wherein the immunotherapy-associated neurotoxicity is immune effector cell-associated neurotoxicity syndrome (ICANS).

46. The method of claim 42, wherein the immunotherapy comprises an immune effector cell therapy.

47. The method of claim 46, wherein the immune effector cell therapy is a CAR T cell therapy selected from engineered CAR T, universal allogeneic CAR T, CD19-specific CAR T, anti-CD19 CAR T, anti-BCMA CAR T, anti-CD22, anti-CAIX, anti-PSMA, anti-MUC1, anti-FRα, anti-meso-RNA, anti-CEA, anti-IL13Rα2, or anti-HER2.

48. The method of claim 42, wherein the immunotherapy comprises a bispecific antibody therapy.

49. A method of preventing immunotherapy-associated neurotoxicity in a subject in need thereof, the method comprising:
providing a biological sample from the subject, wherein the biological sample is obtained before the subject is expected to receive an immunotherapy;
measuring a neurofilament light chain (NfL) level in the biological sample; and
administering a treatment for the immunotherapy-associated neurotoxicity before, after, and/or during immunotherapy when the subject has an elevated NfL level compared to a control or when the subject has an NfL level of more than about 44 pg/m L.

50. The method of claim 49, wherein the biological sample comprises serum, plasma, or cerebrospinal fluid (CSF).

51. The method of claim 49, wherein measuring the NfL level in the biological sample is performed up to 30 days before the subject is expected to receive immunotherapy.

52. The method of claim 49, wherein the immunotherapy-associated neurotoxicity is immune effector cell-associated neurotoxicity syndrome (ICANS).

53. The method of claim 49, wherein the immunotherapy comprises an immune effector cell therapy.

54. The method of claim 53, wherein the immune effector cell therapy is a CAR T cell therapy selected from engineered CAR T, universal allogeneic CAR T, CD19-specific CAR T, anti-CD19 CAR T, anti-BCMA CAR T, anti-CD22, anti-CAIX, anti-PSMA, anti-MUC1, anti-FRα, anti-meso-RNA, anti-CEA, anti-IL13Rα2, or anti-HER2.

55. The method of claim 49, wherein the immunotherapy comprises a bispecific antibody therapy.
